(19) United States
(12) Patent Application Publication
(10) Pub. No.: US 2026/0337690 A1
(43) Pub. Date: Oct. 29, 2026

(54) Systems and Methods for Cancer Screening

(71) Applicants: The Board of Trustees of the Leland Stanford Junior University, Stanford, CA (US); IHC Health Services, Inc., Salt Lake City, UT (US)

(72) Inventors: Hanlee P. Ji, Stanford, CA (US); Billy Tsz Cheong Lau, Palo Alto, CA (US); Lincoln Nadauld, Santa Clara, UT (US)

(73) Assignees: The Board of Trustees of the Leland Stanford Junior University, Stanford, CA (US); IHC Health Services, Inc., Salt Lake City, UT (US)

(21) Appl. No.: 19/101,555

(22) PCT Filed: Aug. 7, 2023
(86) PCT No.: PCT/US2023/071782

§ 371 Date: Feb. 5, 2025

Related U.S. Application Data
(60) Provisional application No. 63/370,638, filed on Aug. 5, 2022.
(60) Provisional application No. 63/402,834, filed on Aug. 31, 2022.

(51) Int. Cl. C12Q 1/6886; G16B 40/00

(52) U.S. Cl. CPC C12Q 1/6886; G16B 40/00; C12Q 2600/154

(57) ABSTRACT

Systems and methods for cancer screening are provided. The system and methods can utilize cell-free DNA (cfDNA) to identify one or more cfDNA features. The cfDNA features can include sequence variant data features, base modification data features, and cfDNA molecule fragment length data features. A trained machine-learning model can utilize cfDNA features to determine whether an individual has a cancer.

### CROSS-REFERENCE TO RELATED APPLICATIONS

The current application claims priority under 35 U.S.C. § 119(e) to U.S. Provisional Application Ser. No. 63/402,834, entitled “Methods and Systems for Breast Cancer Screening” to Ji et al., filed Aug. 31, 2022, and to U.S. Provisional Application Ser. No. 63/370,638, entitled “Methods and Systems for Breast Cancer Screening” to Ji et al., filed Aug. 5, 2022, the disclosures of which are incorporated herein by reference in their entirety.

### TECHNOLOGICAL FIELD

The disclosure is generally directed to systems and methods for cancer screening using cell free DNA.

### BACKGROUND

Current methods of diagnosing breast cancer include medical imaging, such as ultrasound and mammography. However, imaging methods require advanced scheduling, require a time commitment, and can be uncomfortable. For these reasons, many individuals may not comply or participate in imaging-based cancer screening. Delayed detection of cancer can lead to poorer outcomes and prognoses.

Minimally invasive blood tests that can detect somatic alterations (e.g., mutated nucleic acids) based on the analysis of cell free nucleic acids (e.g., cfDNA) are attractive candidates for cancer screening applications due to the relative ease of obtaining biological samples (e.g., liquid biopsy). Cell free tumor nucleic acid molecules can be utilized as a sensitive and specific biomarker in numerous cancer subtype, but the utility of such cell free nucleic methods has remained limited.

### SUMMARY

This summary is meant to provide some examples and is not intended to be limiting of the scope of the invention in any way. For example, any feature included in an example of this summary is not required by the claims, unless the claims explicitly recite the features.

In some implementations, a method is for performing a diagnostic scan for cancer. The method comprises obtaining a cell-free DNA sample of an individual. The cell-free DNA sample comprises a plurality of originating cell-free DNA molecule fragments. The method comprises sequencing, using a single molecule sequencing platform, the cell-free DNA sample to yield a sequencing result. The method comprises identifying, using a computational processing system, one or more cell-free DNA features within the sequencing result. The method comprises entering, using the computational processing system, the one or more cell-free DNA features within one or more machine-learning models trained to detect the presence of cell-free DNA molecules derived from a cancer within the cell-free DNA sample. The method comprises determining, using the computational processing system, whether the cell-free DNA sample contains cell-free DNA molecules derived from a cancer based entering of the one or more cell-free DNA features within the one or more machine-learning models.

In some implementations, the single molecule sequencing platform is selected from Oxford Nanopore Technologies PromethION sequencing platform, Oxford Nanopore Technologies MinION sequencing platform, Oxford Nanopore Technologies GridION sequencing platform, or Pacific Bioscience's Single Molecule, Real-Time sequencing platform.

In some implementations, the cell-free DNA sample comprises greater than 10,000 originating cell-free DNA molecule fragments.

In some implementations, the one or more cell-free DNA features comprises at least one of: a base modification data feature or a cfDNA molecule fragment length data feature.

In some implementations, the one or more cell-free DNA features comprises both the base modification data feature and the cfDNA molecule fragment length data feature.

In some implementations, the base modification data feature comprises at least one of: presence of base modifications, fraction of base modifications, base modifications associated with cancer, or patterns of base modifications associated with cancer.

In some implementations, base modification features comprise at least one of: cytosine methylation status at various loci, fraction of cytosines methylated, and patterns of cytosines methylated.

In some implementations, the cytosine methylation status at various loci is associated with a cancer, the fraction of cytosines methylated is associated with a cancer, and the patterns of cytosines methylated is associated with a cancer.

In some implementations, the cell-free DNA molecule fragment length data feature comprises at least one of: a frequency of a particular molecule fragment length, a frequency of molecule fragment length within a range, a variability of molecule fragment lengths within the cell-free DNA sample.

In some implementations, the one or more machine learning models is trained to further determine whether the cell-free sample has a cancer-related characteristic.

In some implementations, the cancer-related characteristic is one of: cell of origin, tissue of origin, morphology, cancer subtype, or cancer stage.

In some implementations, the one or more machine learning models comprises an ensemble model.

In some implementations, the one or more machine learning models comprises one of: LASSO regression, ridge regression, k-nearest neighbors, elastic net, least angle regression (LAR), random forest regression, support vector machines (SVMs), decision trees, random forests, or naïve Bayes.

In some implementations, the method further comprises extracting a biological sample from the individual. The biological sample comprises cell-free DNA. The method further comprises.

In some implementations, processing the biological sample to yield the cell-free DNA sample.

In some implementations, the biological sample is one of: plasma, blood, lymph, saliva, urine, stool, cerebral spinal fluid, or mucus.

In some implementations, the method further comprises determining, using the computational processing system, that the cell-free DNA sample contains cell-free DNA molecules derived from a cancer. The method further comprises performing a clinical intervention.

In some implementations, the clinical intervention is a further clinical evaluation.

In some implementations, the clinical intervention is administration of a treatment.

In some implementations, the cancer is selected from: acute lymphoblastic leukemia (ALL), acute myeloid leukemia (AML), anal cancer, astrocytomas, basal cell carcinoma, bile duct cancer, bladder cancer, breast cancer, Burkitt's lymphoma, cervical cancer, chronic lymphocytic leukemia (CLL) chronic myelogenous leukemia (CML), chronic myeloproliferative neoplasms, colorectal cancer, diffuse large B-cell lymphoma, endometrial cancer, ependymoma, esophageal cancer, esthesioneuroblastoma, Ewing sarcoma, fallopian tube cancer, follicular lymphoma, gallbladder cancer, gastric cancer, gastrointestinal carcinoid tumor, hairy cell leukemia, hepatocellular cancer, Hodgkin lymphoma, hypopharyngeal cancer, Kaposi sarcoma, Kidney cancer, Langerhans cell histiocytosis, laryngeal cancer, leukemia, liver cancer, lung cancer, lymphoma, melanoma, Merkel cell cancer, mesothelioma, mouth cancer, neuroblastoma, non-Hodgkin lymphoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, T-cell lympohoma, testicular cancer, thymoma, thyroid cancer, uterine cancer, vaginal cancer, and vascular tumors.

In some implementations, the diagnostic scan is performed: prior to any indication of cancer, before symptoms of cancer are recognized, to detect if residual cancer exists after a treatment, or during treatment to determine whether the treatment is providing the desired response.

### BRIEF DESCRIPTION OF THE DRAWINGS

The description and claims will be more fully understood with reference to the following figures and data graphs, which are presented as examples of the disclosure and should not be construed as a complete recitation of the scope of the disclosure.

FIG. 1A provides a schematic of tumor cells releasing DNA into circulation.

FIG. 1B provides an example of a computational method to determine the presence of cancer based on cfDNA features.

FIG. 2A provides an example of a computational method to train a machine learning model, select features, and assess the machine learning model.

FIG. 2B provides a schematic of leave-one-out cross validation.

FIG. 3 provides an example of a computational processing system.

FIG. 4 provides an example of a computational network of a distributed computational system.

### DETAILED DESCRIPTION

Turning now to the drawings and data, systems and methods for cancer screening using cell free DNA (cfDNA) are provided in accordance with the various embodiments of the disclosure. In some embodiments, a cfDNA sample is extracted from an individual and utilized to identify a number features. Features can include cfDNA methylation patterns and/or cfDNA fragment size. In some embodiments, the identified features are utilized within a trained machine-learning model to determine whether the cfDNA originated from a cancer. Detection of cancer via the cfDNA can be utilized to perform various clinical assessments and/or treatments.

Conventional methods to assess cfDNA typically comprise bulk deep sequencing of bulk cfDNA from the sample. Conventional sequencing methods that utilize deep sequencing platforms (e.g., Illumina, Roche 454, IonTorrent) require large quantities of nucleic acid molecules as input into the sequencing instrument itself. These conventional methods rely on amplification of the cfDNA via polymerase chain reaction (PCR), whole genome amplification (WGA), or another amplification method prior to sequencing. In order to detect modifications (e.g., methylation) of cfDNA molecules, a conversion reaction (e.g., bisulfite conversion) is performed prior to amplification and sequencing. Bisulfite treatment and other conversion reactions, however, damages DNA, which results in fragmentation, DNA loss, and biased sequencing data.

Here, systems and methods utilize single-molecule sequencing (also referred to as nanopore sequencing) to detect modifications to cfDNA. Single molecule sequencing platforms are capable of reading a nucleic acid sequence, base modification, and fragment length simultaneously. Additionally, such platforms can use smaller quantities of nucleic acids, such as in the nanogram (ng) range, including approximately 4-5 ng (e.g., 4-5±1 ng). As such, the single molecule sequencing platforms can identify cfDNA methylation patterns and cfDNA fragmentation. Additionally, because amplification is not required, each sequencing read is unique to a particular cfDNA molecule, facilitating cfDNA sample analysis.

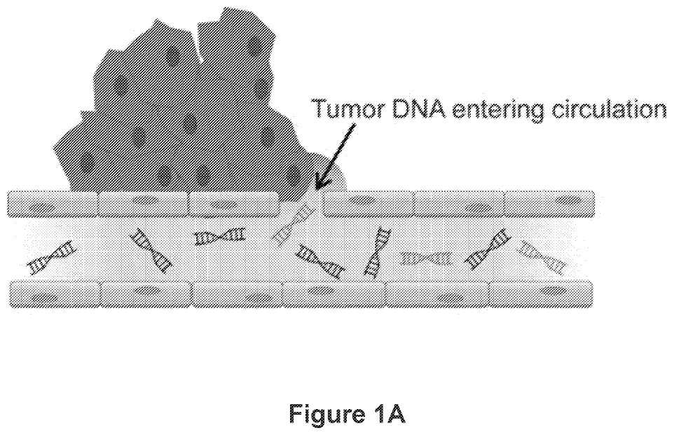

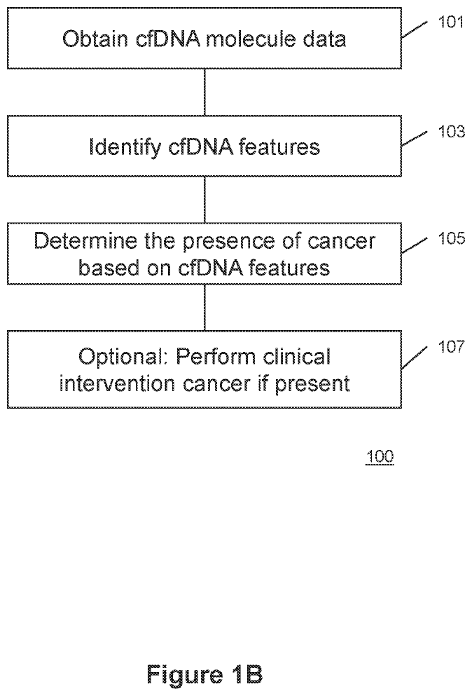

Provided in FIG. 1A is a schematic that demonstrates how cancer can be detected via cfDNA. Due to the rapid overgrowth, cancerous cells rupture or otherwise die, shedding their DNA into the bloodstream of an individual, yielding cfDNA. Tumor derived cfDNA is also referred to as circulating tumor DNA (ctDNA)). A “liquid biopsy” (i.e., a blood sample) can be extracted from the individual via a minimally invasive technique. In some implementations, plasma containing cfDNA is separated from the blood draw. The cfDNA from the blood extraction can be utilized for cancer detection, screening, and prognosis. The use of liquid biopsies facilitates better patient compliance, faster screening, and earlier detection of cancer. Such benefits are especially important for early detection and/or minimal residual disease (MRD) detection, such as when an individual is not showing dramatic symptoms of cancer.

Due to the low input and ability to generate sequence and modification data, various systems and methods of the disclosure utilize single molecule sequencing to identify a cfDNA methylation pattern and/or cfDNA fragmentation of a sample. Such methylation patterns and fragmentation can infer epigenetic information, which can be utilized to detect cancer-derived cfDNA.

In several embodiments, a machine-learning (ML) model is trained to identify cfDNA derived from a cancer in an individual. ML models can be trained using cfDNA features, such as (for example) cfDNA methylation patterns and/or cfDNA fragmentation. ML models can also be trained to identify other cancer-related characteristics that can be important for diagnosis, such as (for example) cell of origin, tissue of origin, morphology, cancer subtype, cancer stage, or any other characteristic of a cancer.

Several embodiments of the disclosure are directed towards systems and methods for screening for cancer. In many embodiments, a cfDNA sample can be collected from a biological sample of an individual, prepared for single molecule sequencing to identify cfDNA features. In some embodiments, the cfDNA features can be utilized within one or more trained ML models to determine whether the cfDNA sample includes cfDNA that originated from a cancerous cell, indicating the presence or absence of cancer. In some implementations, the one or more trained ML models further determine cell of origin, tissue of origin, morphology, cancer stage, or any other characteristic of a cancer. In some embodiments, when a cancer is detected, the individual undergoes further clinical evaluation and/or is treated for the cancer.

Provided in FIG. 1B is an example of a computational method that can be utilized as a screening diagnostic for cancer. Method 100 can begin by obtaining (101) cfDNA molecule data, which can be obtained by sequencing cfDNA utilizing a single molecule sequencing platform. Single molecule sequencing can capture various cfDNA molecule data, such as chemical modification data (e.g., methylation patterns) and cfDNA fragment length variability (among other molecule properties), that a conventional deep sequencing platform cannot. Profiling methylation and fragment length variability in cfDNA can aid in cancer detection and identify other cancer characterizations (e.g., tissue-of-origin).

Single molecule sequencing can be performed utilizing a cfDNA sample, which can be extracted from a biological sample, which can be collected using a noninvasive or minimally invasive technique. Biological samples include (but are not limited to) plasma, blood, lymph, saliva, urine, stool, cerebral spinal fluid, mucus, and/or other appropriate bodily fluid or waste product.

Biological samples can be rich sources of cfDNA. For example, a human plasma sample typically contains 0.5 to 10 ng per mL of cfDNA, corresponding to 150 to 3,000 copies of the haploid human genome. Some conditions, such as (for example) cancer and donor transplant rejection, will result in higher levels of circulating cfDNA, with levels of greater than 1000 ng per mL having been detected. cfDNA typically circulates in fragments ranging between 120 to 220 bp, with a maximum peak at about 167 bp. Thus, plasma can typically have from about 15 million to 400 million cfDNA copies per mL, and greater than 40 billion cfDNA copies per mL in some conditions.

In some instances, a particular biological sample source can be utilized for detecting a particular cancer type. For example, it may be advantageous to utilize a cerebral spinal fluid to detect a brain cancer, a saliva sample to detect an oral cancer, a urine sample to detect a kidney, bladder, urological, cervical, or vaginal cancer, or stool sample to detect a colorectal cancer. Further, virtually all cancers have been known to release their DNA into the blood stream and/or lymph and thus these sample can be utilized to detect the vast majority of cancer types.

In some embodiments, a biological sample is collected prior to any indication of cancer. In some embodiments, a biological sample is collected to provide an early screen in order to detect a cancer (e.g., before symptoms of cancer are present or are recognized). In some embodiments, a biological sample is collected to detect if residual cancer (e.g., MRD) exists after a treatment. In some embodiments, a biological sample is collected during treatment to determine whether the treatment is providing the desired response.

Screening of any particular cancer can be performed. Cancers that can be screened include (but are not limited to) acute lymphoblastic leukemia (ALL), acute myeloid leukemia (AML), anal cancer, astrocytomas, basal cell carcinoma, bile duct cancer, bladder cancer, breast cancer, Burkitt's lymphoma, cervical cancer, chronic lymphocytic leukemia (CLL) chronic myelogenous leukemia (CML), chronic myeloproliferative neoplasms, colorectal cancer, diffuse large B-cell lymphoma, endometrial cancer, ependymoma, esophageal cancer, esthesioneuroblastoma, Ewing sarcoma, fallopian tube cancer, follicular lymphoma, gallbladder cancer, gastric cancer, gastrointestinal carcinoid tumor, hairy cell leukemia, hepatocellular cancer, Hodgkin lymphoma, hypopharyngeal cancer, Kaposi sarcoma, Kidney cancer, Langerhans cell histiocytosis, laryngeal cancer, leukemia, liver cancer, lung cancer, lymphoma, melanoma, Merkel cell cancer, mesothelioma, mouth cancer, neuroblastoma, non-Hodgkin lymphoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, T-cell lymphoma, testicular cancer, thymoma, thyroid cancer, uterine cancer, vaginal cancer, and vascular tumors.

Cell-free nucleic acids can be prepared, isolated, and/or purified from a biological sample by any appropriate means. In some embodiments, cells are removed from a biological sample or otherwise separated from cfDNA. For example, plasma can be isolated from blood via centrifugation to separate cfDNA from blood cells. In some embodiments, column purification is utilized to purify cfDNA from sample source (e.g., QIAamp Circulating Nucleic Acid Kit from Qiagen, Hilden, Germany).

In several embodiments, cfDNA is sequenced using a single molecule sequencing platform (also referred to as nanopore sequencing) to yield cfDNA molecule data. Any appropriate single molecule sequencing platform can be utilized, such as (for example) Oxford Nanopore Technologies PromethION, MinION, and GridION sequencing platforms (Oxford, UK) or Pacific Bioscience's Single Molecule, Real-Time (SMRT) sequencing platform (Menlo Park, CA).

Any appropriate concentration or number of cfDNA molecule can be utilized for sequencing. A cfDNA sample of originating nucleic acid molecule fragments for a sequencing reaction can have greater than 10,000 originating nucleic acid molecule fragments, greater than 100,000 originating nucleic acid molecule fragments, greater than 1,000,000 originating nucleic acid molecule fragments, greater than 10,000,000 originating nucleic acid molecule fragments, greater than 100,000,000 originating nucleic acid molecule fragments, or greater than 1,000,000,000 originating nucleic acid molecule fragments.

Method 100 further identifies (103) cfDNA features from the single molecule sequencing result. A cfDNA feature is any data feature that can identified within and/or extracted from the single molecule sequencing result. Cell-free DNA features include (but are not limited to) sequence variant data features, base modification data features, and cfDNA molecule fragment length data features. Sequence variant data features include (but are not limited to) presence of nucleic acid variants, fraction of nucleic acid variants, and nucleic acid variants associated with cancer (or particular cancer types or subtypes). Nucleic acid variants include (but are not limited to) single nucleotide variants (SNVs), insertions, deletions, and transversions. Base modification features include (but are not limited to) presence of base modifications (both generally and/or at particular residues), fraction of base modifications (e.g., fraction of methylated cytosines), base modifications (and patterns thereof) associated with cancer (or particular cancer types or subtypes). Modified nucleobases include (but are not limited to) 5-methylcytosine (5mC), 5-hydroxymethylcytosine (5hmC), β-glucosyl-5-hydroxymethylcytosine (5gmC), 5-formylcytosine (5fC), and N6-methyladenine (6mA). Cell-free DNA molecule fragment length data features include (but are not limited to) fraction of particular (or range of) cfDNA molecule fragment lengths and variability of particular (or range of) cfDNA molecule fragment lengths.

Cell-free DNA features can be identified utilizing the sequencing result by any appropriate means. Sequencing results can be processed, filtered, and/or aligned with a reference genome to call out cfDNA data features. For example, in some implementations, cfDNA fragment molecules reads with greater than 800 bp are removed from analysis, which is indicate higher molecular weight DNA that is likely contamination derived from lysed cells within the original biological sample. In various implementations, cfDNA fragment molecules reads are removed from analysis if greater than 400 bp, if greater than 500 bp, if greater than 600 bp, if greater than 700 bp, if greater than 800 bp, if greater than 900 bp, or if greater than 1000 bp.

Cell-free DNA features can be identified by assessing the sequencing reads within the sequencing result. In some implementations, at least 50% of the sequencing reads are assessed to identify cell-free DNA features. In some implementations, at least 60% of the sequencing reads are assessed to identify cell-free DNA features. In some implementations, at least 70% of the sequencing reads are assessed to identify cell-free DNA features. In some implementations, at least 80% of the sequencing reads are assessed to identify cell-free DNA features. In some implementations, at least 90% of the sequencing reads are assessed to identify cell-free DNA features. In some implementations, at least 95% of the sequencing reads are assessed to identify cell-free DNA features. In some implementations, at least 99% of the sequencing reads are assessed to identify cell-free DNA features.

Base modification features can include cytosine methylation status at various loci, fraction of cytosines methylated, and patterns of cytosines methylated. One example of a methylation status feature is the frequency of a particular cytosine residue modified within a sample (i.e., number of cfDNA molecules that comprise a modification at particular residue). Various examples of fraction of cytosines methylated is number of cytosines methylated within a particular region, per number of bases, and/or on an originating cfDNA molecule. A region can be defined by any useful way. Regions can be defined by exonic regions, intronic regions, a sequence of one or more genes, one or more regulatory regions, one or more CpG islands, and/or as determined by a user. A region can be defined by CpG frequency (e.g. sequences with a density CpG over or under a threshold). Various examples of patterns of cytosines methylated can include a frequency that two or more particular cytosines are simultaneously methylated and a frequency that two or more particular CpG islands are simultaneously methylated. Methylation frequency and patterns can be assessed on originating cfDNA molecule level and/or at a sample level. Although cytosine methylation is used as an example, it should be understood that any base modification can be utilized for identifying base modification features.

Cell-free DNA molecule fragment length data features can be assessed at a sample level. In some implementations, the frequency of a particular molecule fragment length or range of lengths can be utilized a feature. In some implementations, the variability of molecule fragment lengths within a sample can be utilized as a feature. It has been discovered that the cancer-derived cfDNA fragments exhibit greater variability than those derived from non-cancer cells.

The utility of cfDNA features can be determined empirically from clinical data comparing sequencing results between individuals having a cancer and controls (i.e., individuals lacking cancer). The empirical data can be derived from cfDNA sources and/or cellular sources (e.g., tumor DNA). The empirical data can further be derived from any form of methylation sequencing (e.g., inclusive of bisulfite sequencing). In some implementations, the empirical data is derived in a manner that is similar to that of method 100 (i.e., cfDNA source and single molecule sequencing). The significance can further be determined using a machine learning model, which can be trained to delineate individuals having a cancer and controls and identify which data features provide the best classification and/or regression score.

Method 100 also determines (105) presence of cancer in an individual based on the identified cfDNA features, which can be determined using one or more trained ML models. In some embodiments, one or more cfDNA features are utilized within a trained computational that has been trained to classify and/or score a likelihood that a biological sample includes cfDNA molecules derived from a cancer. The one or more ML models can include models that further determine various cancer-related characteristics, including (but not limited to) cell of origin, tissue of origin, morphology, cancer subtype, and cancer stage.

Any appropriate machine learning model and architecture can be utilized. In some implementations, multiple trained machine models are utilized and/or combined (e.g., an ensemble model). ML models that can be implemented include (but are not limited to) regression-based and/or classification-based models. Generally, regression-based models provide a score that indicates a likelihood of the cancer whereas a classification-based model classifies a sample as likely to include or to not include cancer. Regression-based models include (but are not limited to) LASSO regression, ridge regression, k-nearest neighbors, elastic net, least angle regression (LAR), and random forest regression. Classification-based models include (but are not limited to) support vector machines (SVMs), decision trees, random forests, and naïve Bayes. In some embodiments, a regression-based model or a classification-based model is regularized, while in various embodiments, a regression-based model or a classification-based model is gradient boosted.

Method 100 can optionally perform (107) a clinical intervention when the ML model indicates that the cfDNA sample contains cfDNA molecules derived from a cancer. Clinical interventions can include further clinical evaluation of or administration of a treatment to an individual. In some embodiments, a clinical evaluation is performed, such as (for example) a blood test, medical imaging, physical exam, a tumor biopsy, or any combination thereof. In some embodiments, a clinical evaluation comprises performing a diagnostic to determine a stage of cancer. In some embodiments, a treatment is administered, such as (for example) surgery, chemotherapy, radiotherapy, immunotherapy, hormone therapy, targeted drug therapy, medical surveillance, or any combination thereof. In some embodiments, an individual is assessed and/or treated by medical professional, such as a doctor, nurse, dietician, or similar.

While a specific example of a computational method for determining the presence of cancer is described above, one of ordinary skill in the art can appreciate that various steps of the method can be performed in different orders and that certain steps may be optional according to some embodiments of the disclosure. As such, it should be clear that the various steps of the process could be used as appropriate to the requirements of specific applications. Furthermore, any of a variety of computational methods for determining the presence of cancer appropriate to the requirements of a given application can be utilized in accordance with various embodiments of the disclosure.

### Model Training and Feature Selection

Several embodiments are directed towards training a ML model to determine the presence of cancer based on cfDNA features. In many embodiments, the model is trained such that it can be utilized in a cancer diagnostic screen utilizing a cfDNA sample and single molecule sequencing platform. A ML model can utilize one or more cfDNA features. Accordingly, various embodiments are directed to the selection of one or more cfDNA features to be utilized within a ML model trained to determine the presence of cancer.

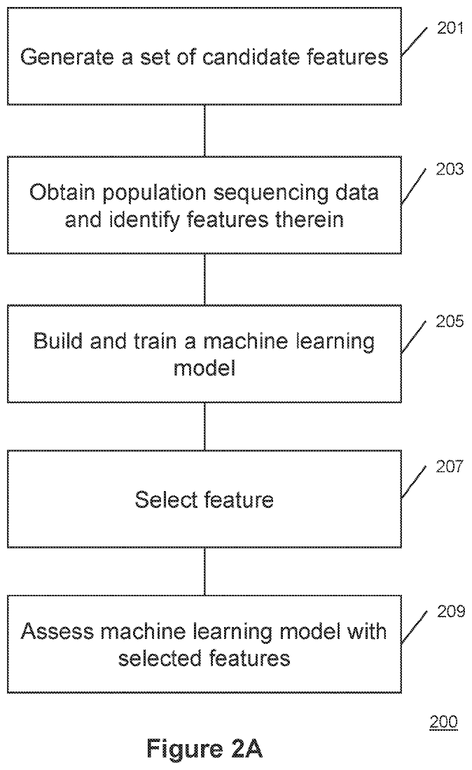

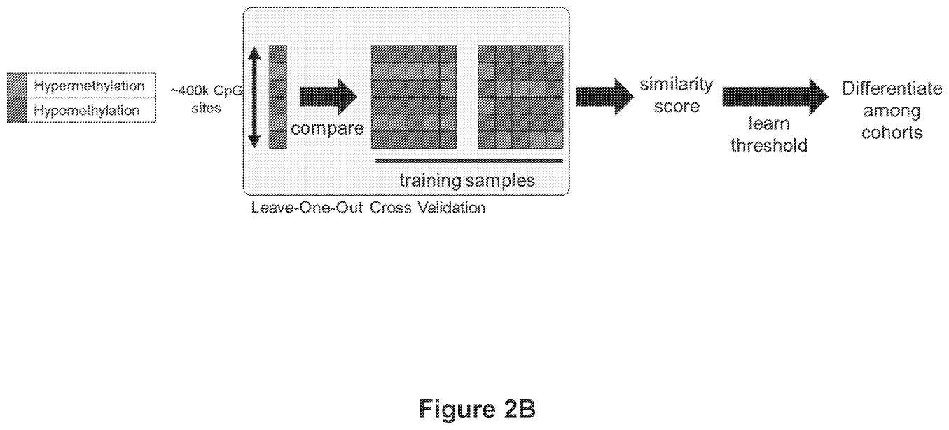

Provided in FIG. 2A is an example of a method for prioritizing cfDNA features. A cfDNA feature is any data feature that can identified within and/or extracted from the single molecule sequencing result. Cell-free DNA features include (but are not limited to) sequence variant data features, base modification data features, cfDNA molecule fragment length data features. Sequence variant data features include (but are not limited to) presence of nucleic acid variants, fraction of nucleic acid variants, and nucleic acid variants associated with cancer (or particular cancer types or subtypes). Nucleic acid variants include (but are not limited to) single nucleotide variants (SNVs), insertions, deletions, and transversions. Base modification features include (but are not limited to) presence of base modifications (both generally and/or at particular residues), fraction of base modifications (e.g., fraction of methylated cytosines), base modifications (and patterns thereof) associated with cancer (or particular cancer types or subtypes). Modified nucleobases include (but are not limited to) 5-methylcytosine (5mC), 5-hydroxymethylcytosine (5hmC), β-glucosyl- 5-hydroxymethylcytosine (5gmC), 5-formylcytosine (5fC), and N6-methyladenine (6mA). Cell-free DNA molecule fragment length data features include (but are not limited to) fraction of particular (or range of) cfDNA molecule fragment lengths and variability of particular (or range of) cfDNA molecule fragment lengths.

Base modification features can include cytosine methylation status at various loci, fraction of cytosines methylated, and patterns of cytosines methylated. One example of a methylation status feature is the frequency of a particular cytosine residue modified within a sample (i.e., number of cfDNA molecules that comprise a modification at particular residue). Various examples of fraction of cytosines methylated is number of cytosines methylated within a particular region, per number of bases, and/or on an originating cfDNA molecule. A region can be defined by any useful way. Regions can be defined by exonic regions, a sequence of one or more genes, one or more regulatory regions, one or more CpG islands, and/or as determined by a user. A region can be defined by CpG frequency (e.g. sequences with a density CpG over or under a threshold). Various examples of patterns of cytosines methylated can include a frequency that two or more particular cytosines are simultaneously methylated and a frequency that two or more particular CpG islands are simultaneously methylated. Methylation frequency and patterns can be assessed on originating cfDNA molecule level and/or at a sample level. Although cytosine methylation is used as an example, it should be understood that any base modification can be utilized for identifying base modification features.

Cell-free DNA molecule fragment length data features can be assessed at a sample level. In some implementations, the frequency of a particular molecule fragment length or range of lengths can be utilized a feature. In some implementations, the variability of molecule fragment lengths within a sample can be utilized as a feature. It has been discovered that the cancer-derived cfDNA fragments exhibit greater variability than those derived from non-cancer cells.

Method 200 can begin by generating (201) a set of candidate features. Cell-free DNA features can be identified utilizing a cfDNA sequencing result by any appropriate means. Sequencing results can be processed, filtered, and/or aligned with a reference genome to call out cfDNA data features. For example, in some implementations, cfDNA fragment molecules reads with greater than 800bp are removed from analysis, which is indicate higher molecular weight DNA that is likely contamination derived from lysed cells within the original biological sample. In various implementations, cfDNA fragment molecules reads are removed from analysis if greater than 400 bp, if greater than 500 bp, if greater than 600 bp, if greater than 700 bp, if greater than 800 bp, if greater than 900 bp, or if greater than 1000 bp.

Method 200 also obtains (203) population sequencing data and identifies cfDNA features therein. The obtained sequencing data can comprise sequencing data from two or more different cohorts, each individual within cohort sharing a particular medical trait. In many embodiments, the sequencing data comprises data of a cohort afflicted with a cancer and a control cohort (i.e., free of cancer). In some embodiments, the two or more cohorts can be delineated by a cancer-related characteristic. Examples of cancer-related characteristics include (but are not limited to) cell of origin, tissue of origin, morphology, cancer subtype, and cancer stage. In one example, a cohort of breast cancer individuals can be delineated by cancer subtypes, such as (for example) luminal A, luminal B, HER2-positive, and triple negative breast cancer (TNBC).

Method 200 also builds and trains (205) a machine learning model to differentiate cfDNA samples among the two or more cohorts (e.g., cancer vs. control). Any method to train a ML model can be utilized. Generally, cfDNA features are identified from the population sequencing data and assigned according to the cohort the feature data was derived from. The ML model can utilize the population data for one or more cfDNA features to learn to differentiate the two or more cohorts. In some implementations, a leave-one-out cross validation (LOOCV) machine-learning model is used to build and train a model. In each LOOCV round, the model is iteratively trained on all samples except for one sample that left out. Model performance can be evaluated on the left-out sample. LOOCV training is attractive because it reduces overfitting and provides a more accurate assessment of the overall stability. See FIG. 2B for a schematic on LOOCV training.

Any appropriate machine learning model and architecture can be utilized. In some implementations, multiple trained machine models are utilized and/or combined (e.g., an ensemble model). ML models that can be implemented include (but are not limited to) regression-based and/or classification-based models. Generally, regression-based models provide a score that indicates a likelihood of the cancer whereas a classification-based model classifies a sample as likely to include or to not include cancer. Regression-based models include (but are not limited to) LASSO regression, ridge regression, k-nearest neighbors, elastic net, least angle regression (LAR), and random forest regression. Classification-based models include (but are not limited to) support vector machines (SVMs), decision trees, random forests, and naïve Bayes. In some embodiments, a regression-based model or a classification-based model is regularized, while in various embodiments, a regression-based model or a classification-based model is gradient boosted.

Method 200 also selects (207) cfDNA features to yield a robust model for classifying and/or predicting the likelihood that a cfDNA sample includes cfDNA derived from a cancer. Features can also be selected for classifying and/or predicting the likelihood that a cancer has a cancer-related characteristic.

We performed a feature selection step to discover informative CpG sites for building multiple types of statistical scores in each LOOCV round. The first type was statistically significant CpG sites between case and control: we applied FDR-corrected statistical testing to identify the differentially methylated CpG sites across whole genome between case and control groups. The second type was case-specific CpG sites; these were sites that were found to have read coverage in only case samples and no control samples. The third type of informative site was control-specific CpG sites. These were sites that were found to have read coverage in only control samples and no case samples. The final score considers all CpG sites. For each informative CpG site, we also record information about the extent of methylation for the case and control group.

In one example, cfDNA cytosine methylation features can be evaluated and selected. A statistical test can be performed for each CpG site in order to determine those with statistically significant methylation for differentiating among the two or more cohorts. Each sample's methylation bedgraph file can be merged using the bedtools unionbedg command. This created an aggregated matrix where the rows are the union of all observed CpG sites, columns are each sequenced sample, and the values of the matrix correspond to the methylation value.

To select for statistically significant methylation sites, Welch's t-test can be applied to the methylation values among the two or more cohorts. Subsequently, FDR-based multiple testing correction on all the p-values was performed. Sites with adjusted p-values that were less than a threshold (e.g., 1e-5) after FDR correction can be used. For each significant site, its corresponding genomic coordinate, adjusted p-value, and associated group-wise methylation averages can be recorded.

In one example, physical features of sequenced cfDNA can be automatically extracted as the statistics of the estimated size distribution of cfDNA molecules for each sample. To do so, the aggregate distribution of the aligned sequences for each sample was fitted to a two-component normal distribution. This yields six fitted features associated with each sample. A random forest model (or similar model) can be trained based on those cfDNA molecule fragment length features, evaluating all samples using LOOCV.

It was found that the sizes of mono-and di-nucleosome cfDNA fragments derived from cancer exhibit greater variability than those derived from non-cancers. To obtain the cfDNA molecule fragment length features involved in nucleosomes, a statistical approach can be developed to estimate the mixture distributions of cfDNA fragments and extract their statistics for each sample. To ensure the accurate estimation, the outlier fragments with reads greater than a threshold (e.g., 800 bp) can be filtered out. The Expectation-Maximization (EM) algorithm can be used to fit a 2-component mixture normal distribution on the remaining fragments. The estimated mixture normal distributions reflect statistical properties of the sizes of mono-nucleosome and di-nucleosome. The initial mean parameters of mono-and bi-nucleosome can be set as 167 bp and 330 bp, respectively. The cfDNA molecule fragment length features of mono-nucleosome and di-nucleosome were performed by the estimated means, variations, and the probabilities from the fitted mixture normal distribution. A matrix where the rows consist of each sample, and columns are the statistics of the cfDNA nucleosome fragments can be generated.

After feature selection, method 200 assesses the ML model with the selected features. Any method for assessing the ability of a ML model can be utilized.

In one example, a ML model can be assessed and scored by mapping the prioritized CpG sites to a given LOOCV validation sample's CpG sites through an intersection on genomic positions, which may be useful when utilizing a sparse sequenced dataset. This intersection procedure accommodates for missing data in contrast to other classification-based methods that rely on imputation. The score can then be calculated by determining the likelihood ratio of a given CpG site's methylation status to match the case cohort and normalized by averaging across all intersected CpG sites. The output of this step is then multiple scores: a score based on intersection with statistically significant CpG sites, a score based on case-specific CpG sites, a score based on control-specific sites, and one based on all CpG sites. Models and features that provide the best scores can be utilized for a diagnostic cancer screening assay.

While a specific example of a computational method for training a model and selecting features is described above, one of ordinary skill in the art can appreciate that various steps of the method can be performed in different orders and that certain steps may be optional according to some embodiments of the disclosure. As such, it should be clear that the various steps of the process could be used as appropriate to the requirements of specific applications. Furthermore, any of a variety of computational methods for training a model and selecting features appropriate to the requirements of a given application can be utilized in accordance with various embodiments of the disclosure.

### Computational Processing System

A computational processing system for determining the presence of cancer in a cfDNA sample in accordance with the various methods of the disclosure typically utilizes a processing system including one or more of a CPU, GPU and/or neural processing engine. In a number of implementations, sequencing results are processed and assessed to detect cfDNA molecules derived from cancer within a sample using a computational processing system. In some implementations, the computational processing system is housed within a computing device associated with sequencer. In some implementations, the computational processing system is housed separately from the sequencer and receives the sequencing results. In certain embodiments, the computational processing system is implemented using a software application on a computing device such as (but not limited to) mobile phone, a tablet computer, and/or portable computer.

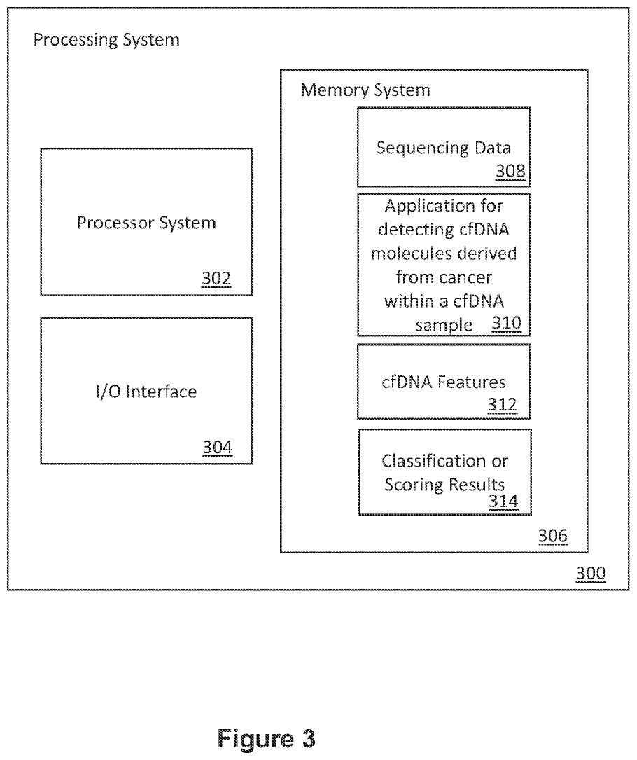

A computational processing system in accordance with various embodiments of the disclosure is illustrated in FIG. 3. The computational processing system 300 includes a processor system 302, an I/O interface 304, and a memory system 306. As can readily be appreciated, the processor system 302, I/O interface 304, and memory system 306 can be implemented using any of a variety of components appropriate to the requirements of specific applications including (but not limited to) CPUs, GPUs, ISPs, DSPs, wireless modems (e.g., WiFi, Bluetooth modems), serial interfaces, depth sensors, IMUs, pressure sensors, ultrasonic sensors, volatile memory (e.g., DRAM) and/or non-volatile memory (e.g., SRAM, and/or NAND Flash). The memory system is capable of storing a sequencing data 308 and an application for detecting cfDNA molecules derived from cancer within a cfDNA sample 310. The application can be downloaded and/or stored in non-volatile memory. When executed, the application for detecting cfDNA molecules derived from cancer within a cfDNA sample is capable of configuring the processing system to implement computational processes including (but not limited to) the computational processes described above and/or combinations and/or modified versions of the computational processes described above. In several embodiments, the detecting cfDNA molecules derived from cancer within a cfDNA sample application 310 trains ML models, selects cfDNA features 312, and can utilize the sequence data 308 to yield a result 314 that classifies or scores the likelihood that a sample includes cfDNA derived from cancer in a sample. In certain implementations, the result 314 is temporarily stored in the memory system during processing and/or saved for use in downstream applications.

While specific computational processing systems are described above with reference to FIG. 3, it should be readily appreciated that computational processes and/or other processes utilized in the provision of assessing modification status in sequencing results in accordance with various embodiments of the disclosure can be implemented on any of a variety of processing devices including combinations of processing devices. Accordingly, computational devices in accordance with the disclosure should be understood as not limited to specific computational processing systems, but can be implemented using any of the combinations of systems described herein and/or modified versions of the systems described herein to perform the processes, combinations of processes, and/or modified versions of the processes described herein.

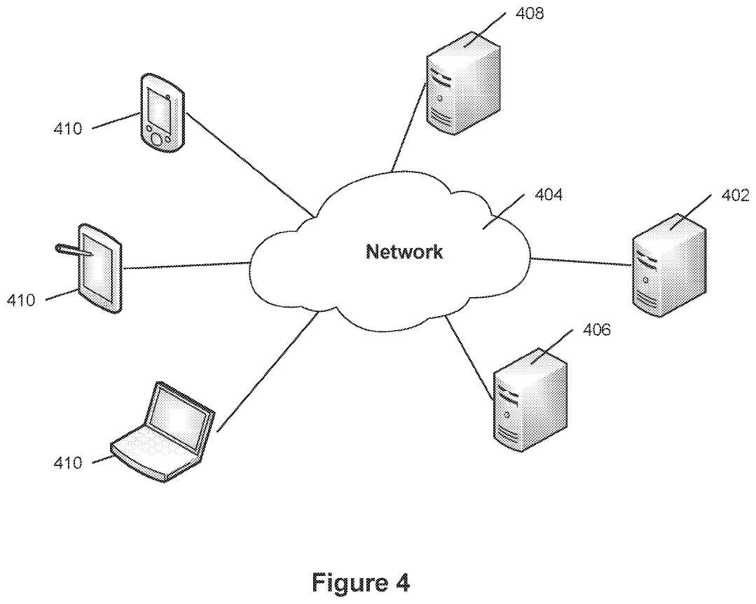

Turning to FIG. 4, an embodiment with distributed computing devices is illustrated. Such embodiments may be useful where computing power is not possible at a local level, and a central computing device (e.g., server) performs one or more features, functions, methods, and/or steps described herein. In such embodiments, a computing device 402 (e.g., server) is connected to a network 404 (wired and/or wireless), where it can receive inputs from one or more computing devices, including cfDNA sequencing data or other relevant information from one or more other remote devices 410. Once computing device 402 performs one or more features, functions, methods, and/or steps described herein, any outputs can be transmitted to one or more computing devices 406, 408, 410 for entering into records, taking medical action—including (but not limited to) clinical assessment and/or therapeutic administration (e.g., immunotherapy, chemotherapy, radiation therapy, etc.)—and/or any other action relevant to a cancer diagnosis or characterization. Such actions can be transmitted directly to a medical professional (e.g., via messaging, such as email, SMS, voice/vocal alert) for such action and/or entered into medical records.

In accordance with still other embodiments, the instructions for the processes can be stored in any of a variety of non-transitory computer readable media appropriate to a specific application.

### Clinical Interventions

Various embodiments are directed towards utilizing detection of cancer to perform clinical interventions. In a number of embodiments, an individual has a liquid or waste biopsy screened and processed by methods described herein to indicate that the individual has cancer and thus an intervention is to be performed. Clinical interventions include clinical evaluations and treatments. Clinical evaluations include (but not limited to) blood tests, medical imaging, physical exams, and tumor biopsies. Treatments include (but not limited to) surgery, chemotherapy, radiotherapy, immunotherapy, hormone therapy, targeted drug therapy, and medical surveillance. In several embodiments, diagnostics are preformed to determine the particular stage of cancer. In some embodiments, an individual is assessed and/or treated by medical professional, such as a doctor, nurse, dietician, or similar.

### Detection of Cancer for Clinical Intervention

In several embodiments as described herein a cancer can be detected utilizing a sequencing result of cell-free nucleic acids derived from a biological sample. In many embodiments, cancer is detected when the sequencing result includes cfDNA features that when entered into a ML model indicate the presence of cancer. Accordingly, in a number of embodiments, cell-free nucleic acids are extracted, processed, and sequenced, and the sequencing result is analyzed to detect cancer. This process is especially useful in a clinical setting to provide a diagnostic scan.

An example of a procedure for a diagnostic scan of an individual for a cancer is as follows:
extract biological sample from individual
prepare and perform single molecule sequencing of a cfDNA sample derived from the biological sample
identify cfDNA features within the sequencing result and utilize features within a trained ML model to detect the presence of cancer
if cancer is detected, perform a clinical intervention

The diagnostic scan can be performed as part of routine screening or as part of a cancer surveillance effort. In some embodiments, the diagnostic scan is performed prior to any indication of cancer. In some embodiments, the diagnostic scan is performed to provide an early screen in order to detect a cancer (e.g., before symptoms of cancer are present or are recognized). In some embodiments, the diagnostic scan is performed to detect if residual cancer (e.g., MRD) exists after a treatment. In some embodiments, the diagnostic scan is performed during treatment to determine whether the treatment is providing the desired response.

In various embodiments, diagnostic scans can be performed for any neoplasm type, including (but not limited to) acute lymphoblastic leukemia (ALL), acute myeloid leukemia (AML), anal cancer, astrocytomas, basal cell carcinoma, bile duct cancer, bladder cancer, breast cancer, Burkitt's lymphoma, cervical cancer, chronic lymphocytic leukemia (CLL) chronic myelogenous leukemia (CML), chronic myeloproliferative neoplasms, colorectal cancer, diffuse large B-cell lymphoma, endometrial cancer, ependymoma, esophageal cancer, esthesioneuroblastoma, Ewing sarcoma, fallopian tube cancer, follicular lymphoma, gallbladder cancer, gastric cancer, gastrointestinal carcinoid tumor, hairy cell leukemia, hepatocellular cancer, Hodgkin lymphoma, hypopharyngeal cancer, Kaposi sarcoma, Kidney cancer, Langerhans cell histiocytosis, laryngeal cancer, leukemia, liver cancer, lung cancer, lymphoma, melanoma, Merkel cell cancer, mesothelioma, mouth cancer, neuroblastoma, non-Hodgkin lymphoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, T-cell lympohoma, testicular cancer, thymoma, thyroid cancer, uterine cancer, vaginal cancer, and vascular tumors.

### Clinical Evaluations and Treatments

A number of embodiments are directed towards performing a diagnostic scan on cell-free nucleic acids of an individual and then based on results of the scan indicating cancer, performing further clinical evaluation and/or treating the individual.

In accordance with various embodiments, numerous types of neoplasms can be detected, including (but not limited to) acute lymphoblastic leukemia (ALL), acute myeloid leukemia (AML), anal cancer, astrocytomas, basal cell carcinoma, bile duct cancer, bladder cancer, breast cancer, Burkitt's lymphoma, cervical cancer, chronic lymphocytic leukemia (CLL) chronic myelogenous leukemia (CML), chronic myeloproliferative neoplasms, colorectal cancer, diffuse large B-cell lymphoma, endometrial cancer, ependymoma, esophageal cancer, esthesioneuroblastoma, Ewing sarcoma, fallopian tube cancer, follicular lymphoma, gallbladder cancer, gastric cancer, gastrointestinal carcinoid tumor, hairy cell leukemia, hepatocellular cancer, Hodgkin lymphoma, hypopharyngeal cancer, Kaposi sarcoma, Kidney cancer, Langerhans cell histiocytosis, laryngeal cancer, leukemia, liver cancer, lung cancer, lymphoma, melanoma, Merkel cell cancer, mesothelioma, mouth cancer, neuroblastoma, non-Hodgkin lymphoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, T-cell lympohoma, testicular cancer, thymoma, thyroid cancer, uterine cancer, vaginal cancer, and vascular tumors.

In accordance with several embodiments, once a diagnosis of cancer is indicated, a number of follow-up clinical evaluations can be performed, including (but not limited to) physical exam, medical imaging, mammography, endoscopy, stool sampling, pap test, alpha-fetoprotein blood test, CA-125 test, prostate-specific antigen (PSA) test, biopsy extraction, bone marrow aspiration, and tumor marker detection tests. Medical imaging includes (but is not limited to) X-ray, magnetic resonance imaging (MRI), computed tomography (CT), ultrasound, and positron emission tomography (PET). Endoscopy includes (but is not limited to) bronchoscopy, colonoscopy, colposcopy, cystoscopy, esophagoscopy, gastroscopy, laparoscopy, neuroendoscopy, proctoscopy, and sigmoidoscopy.

In accordance with many embodiments, once a diagnosis of cancer is indicated, a number of treatments can be performed, including (but not limited to) surgery, chemotherapy, radiation therapy, immunotherapy, targeted therapy, hormone therapy, stem cell transplant, and blood transfusion. In some embodiments, an anti-cancer and/or chemotherapeutic agent is administered, including (but not limited to) alkylating agents, platinum agents, taxanes, vinca agents, anti-estrogen drugs, aromatase inhibitors, ovarian suppression agents, endocrine/hormonal agents, bisphophonate therapy agents and targeted biological therapy agents. Medications include (but are not limited to) cyclophosphamide, fluorouracil (or 5-fluorouracil or 5-FU), methotrexate, thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, capecitabine, anastrozole, exemestane, letrozole, leuprolide, abarelix, buserlin, goserelin, megestrol acetate, risedronate, pamidronate, ibandronate, alendronate, zoledronate, tykerb, daunorubicin, doxorubicin, epirubicin, idarubicin, valrubicin mitoxantrone, bevacizumab, cetuximab, ipilimumab, ado-trastuzumab emtansine, afatinib, aldesleukin, alectinib, alemtuzumab, atezolizumab, avelumab, axtinib, belimumab, belinostat, bevacizumab, blinatumomab, bortezomib, bosutinib, brentuximab vedoitn, briatinib, cabozantinib, canakinumab, carfilzomib, certinib, cetuximab, cobimetnib, crizotinib, dabrafenib, daratumumab, dasatinib, denosumab, dinutuximab, durvalumab, elotuzumab, enasidenib, erlotinib, everolimus, gefitinib, ibritumomab tiuxetan, ibrutnib, idelalisib, imatinib, ipilimumab, ixazomib, lapatinib, lenvatinib, midostaurin, nectiumumab, neratinib, nilotinib, niraparib, nivolumab, obinutuzumab, ofatumumab, olaparib, loaratumab, osimertinib, palbocicilib, panitumumab, panobinostat, pembrolizumab, pertuzumab, ponatinib, ramucirumab, reorafenib, ribociclib, rituximab, romidepsin, rucaparib, ruxolitinib, siltuximab, sipuleucel-T, sonidebib, sorafenib, temsirolimus, tocilizumab, tofacitinib, tositumomab, trametinib, trastuzumab, vandetanib, vemurafenib, venetoclax, vismodegib, vorinostat, and ziv-aflibercept. In accordance with various embodiments, an individual may be treated, by a single medication or a combination of medications described herein. A common treatment combination is cyclophosphamide, methotrexate, and 5-fluorouracil (CMF).

Many embodiments are directed to diagnostic or companion diagnostic scans performed during cancer treatment of an individual. When performing diagnostic scans during treatment, the ability of agent to treat the cancer growth can be monitored. Most anti-cancer therapeutic agents result in death and necrosis of neoplastic cells, which should release higher amounts nucleic acids from these cells into the samples being tested. Accordingly, the level of circulating-tumor nucleic acids can be monitored over time, as the level should increase during early treatments and begin to decrease as the number of cancerous cells are decreased. In some embodiments, treatments are adjusted based on the treatment effect on cancer cells. For instance, if the treatment isn't cytotoxic to neoplastic cells, a dosage amount may be increased or an agent with higher cytotoxicity can be administered. In the alternative, if cytotoxicity of cancer cells is good but unwanted side effects are high, a dosage amount can be decreased or an agent with less side effects can be administered.

Various embodiments are also directed to diagnostic scans performed after treatment of an individual to detect residual disease and/or recurrence of cancer. If a diagnostic scan indicates residual and/or recurrence of cancer, further diagnostic tests and/or treatments may be performed as described herein. If the cancer and/or individual is susceptible to recurrence, diagnostic scans can be performed frequently to monitor any potential relapse.

1. A method of performing a diagnostic scan for cancer, comprising:
obtaining a cell-free DNA sample of an individual, wherein the cell-free DNA sample comprises a plurality of cell-free DNA molecule fragments;
sequencing, using a single molecule sequencing platform, the cell-free DNA sample to yield a sequencing result;
identifying, using a computational processing system, one or more cell-free DNA features within the sequencing result;
entering, using the computational processing system, the one or more cell-free DNA features within one or more machine-learning models trained to detect the presence of cell-free DNA molecules derived from a cancer within the cell-free DNA sample; and
determining, using the computational processing system, whether the cell-free DNA sample contains cell-free DNA molecules derived from a cancer based entering of the one or more cell-free DNA features within the one or more machine-learning models.

2. The method of claim 1, the single molecule sequencing platform is selected from Oxford Nanopore Technologies PromethION sequencing platform, Oxford Nanopore Technologies MinION sequencing platform, Oxford Nanopore Technologies GridION sequencing platform, or Pacific Bioscience's Single Molecule, Real-Time sequencing platform.

3. The method of claim 1, wherein the cell-free DNA sample comprises greater than 10,000 cell-free DNA molecule fragments.

4. The method of claim 1, wherein the one or more cell-free DNA features comprises at least one of: a base modification data feature or a cfDNA molecule fragment length data feature.

5. The method of claim 4, wherein the one or more cell-free DNA features comprises both the base modification data feature and the cfDNA molecule fragment length data feature.

6. The method of claim 4, wherein the base modification data feature comprises at least one of: presence of base modifications, fraction of base modifications, base modifications associated with cancer, or patterns of base modifications associated with cancer.

7. The method of claim 6, wherein base modification features comprise at least one of: cytosine methylation status at various loci, fraction of cytosines methylated, and patterns of cytosines methylated.

8. The method of claim 7, wherein the cytosine methylation status at various loci is associated with a cancer, the fraction of cytosines methylated is associated with a cancer, and the patterns of cytosines methylated is associated with a cancer.

9. The method of claim 4, wherein the cell-free DNA molecule fragment length data feature comprises at least one of: a frequency of a particular molecule fragment length, a frequency of molecule fragment length within a range, a variability of molecule fragment lengths within the cell-free DNA sample.

10. The method of claim 1, wherein the one or more machine learning models is trained to further determine whether the cell-free sample has a cancer-related characteristic.

11. The method of claim 10, wherein the cancer-related characteristic is one of: cell of origin, tissue of origin, morphology, cancer subtype, or cancer stage.

12. The method of claim 1, wherein the one or more machine learning models comprises an ensemble model.

13. The method of claim 1, wherein the one or more machine learning models comprises one of: LASSO regression, ridge regression, k-nearest neighbors, elastic net, least angle regression (LAR), random forest regression, support vector machines (SVMs), decision trees, random forests, or naïve Bayes.

14. The method of claim 1 further comprising:
extracting a biological sample from the individual, wherein the biological sample comprises the cell-free DNA molecule fragments; and
processing the biological sample to yield the cell-free DNA sample.

15. The method of claim 14, wherein the biological sample is one of: plasma, blood, lymph, saliva, urine, stool, cerebral spinal fluid, or mucus.

16. The method of claim 1 further comprising:
determining, using the computational processing system, that the cell-free DNA sample contains cell-free DNA molecule fragments derived from a cancer; and
performing a clinical intervention.

17. The method of claim 16, wherein the clinical intervention is a further clinical evaluation.

18. The method of claim 16, the clinical intervention is administration of a treatment.

19. The method of claim 1, wherein the cancer is selected from: acute lymphoblastic leukemia (ALL), acute myeloid leukemia (AML), anal cancer, astrocytomas, basal cell carcinoma, bile duct cancer, bladder cancer, breast cancer, Burkitt's lymphoma, cervical cancer, chronic lymphocytic leukemia (CLL) chronic myelogenous leukemia (CML), chronic myeloproliferative neoplasms, colorectal cancer, diffuse large B-cell lymphoma, endometrial cancer, ependymoma, esophageal cancer, esthesioneuroblastoma, Ewing sarcoma, fallopian tube cancer, follicular lymphoma, gallbladder cancer, gastric cancer, gastrointestinal carcinoid tumor, hairy cell leukemia, hepatocellular cancer, Hodgkin lymphoma, hypopharyngeal cancer, Kaposi sarcoma, Kidney cancer, Langerhans cell histiocytosis, laryngeal cancer, leukemia, liver cancer, lung cancer, lymphoma, melanoma, Merkel cell cancer, mesothelioma, mouth cancer, neuroblastoma, non-Hodgkin lymphoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, T-cell lympohoma, testicular cancer, thymoma, thyroid cancer, uterine cancer, vaginal cancer, and vascular tumors.

20. The method of claim 1, wherein the diagnostic scan is performed: prior to any indication of cancer, before symptoms of cancer are recognized, to detect if residual cancer exists after a treatment, or during treatment to determine whether the treatment is providing the desired response.
